(19) United States
(12) Patent Application Publication
(10) Pub. No.: US 2007/0060874 A1
(43) Pub. Date: Mar. 15, 2007

(54) APPARATUS AND METHODS FOR CONTROLLING AND AUTOMATING FLUID INFUSION ACTIVITIES

(76) Inventors: Matthew T. Nesbitt, Atlanta, GA (US); Ross G. Krogh, Cincinnati, OH (US); Randy R. Stephens, Fairfield, OH (US); Paul J. Niklewski, Cincinnati, OH (US); Michael E. Boehm, Cincinnati, OH (US); Satyajeet V. Parakh, Ichalkaranji (IN)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/530,576

(22) Filed: Sep. 11, 2006

Related U.S. Application Data
(60) Provisional application No. 60/716,308, filed on Sep. 12, 2005.

(51) Int. Cl. A61M 5/14

(52) U.S. Cl. 604/80; 604/251

(57) ABSTRACT

The present invention provides apparatuses and methods to safely and economically deliver infusion fluid to a patient during a medical procedure. The infusion fluid may be a sedative, analgesic, amnestic or other pharmaceutical agent (drug) for alleviating a patient's pain and anxiety before, during and/or after a medical or surgical procedure. In general the apparatus comprises a microprocessor-based controller that receives inputs from a plurality of physiological monitors attached to a patient. The system controller processes the data from the physiological monitors and based upon a fluid infusion algorithm delivers infusion fluid to a patient. The physiological monitors monitor the patient throughout the course of the procedure and depending upon the health of the patient, drug delivery may be adjusted to optimize the procedure while ensuring the patient's health is maintained. Functionality detectors such as an occlusion sensor, air-in-line sensor and a fluid detection sensor alert a clinician to such hazards as a pressure build up in the infusion line, air-bubbles in the infusion line, and the absence of fluid in the infusion line.

### CROSS-REFERENCE TO RELATED APPLICATIONS

The present invention claims priority from U.S. provisional patent application No. 60/716,308 filed on Sep. 12, 2005.

### FIELD OF THE INVENTION

The present invention relates to an apparatus for controlling the administration and infusion of fluids to a patient and for automating activities related toward fluid infusion traditionally performed by a clinician.

### BACKGROUND OF THE INVENTION

In current practice, sedative drugs are delivered to patients undergoing certain medical procedures. Sedative drugs are stored in a reservoir and delivered to a patient by either a gravity feed mechanism or by way of a fluid pump. In some instances, multiple fluids are simultaneously supplied to a patient; in this case, the sedative may be mixed prior to entering the bloodstream or may enter the bloodstream apart from each other.

After the patient has received the sedative drugs, either a clinician or an automated patient monitoring system monitors the patient for a physiological reaction to the infused fluid. Typically physiological parameters such as blood pressure, blood oxygen saturation, temperature, respiration rate, patient alertness and other parameters understood in the art are continuously evaluated to asses what effect the infused fluid is having upon the patient. In current practice, a clinician or patient monitoring device evaluates data provided by physiological monitors and a decision is made as to increase, maintain, decrease or cease the flow of infusion fluid to the patient. After a decision is made, the volume and rate of fluid infusion are then adjusted to achieve a desired patient condition. Care is given to avoid over infusion or under infusion of sedative drugs to a patient as this may endanger the patient.

Others have attempted to address the automation and delivery of sedative drugs with varying degrees of success. As described in the examples below, previous attempts have been directed toward two approaches to automating infusion. The first approach being a very simple device for delivering a sedative drug to a patient with limited feedback and safety functions and the second approach involves a complex device with many feedback and safety functions.

U.S. Pat. No. 6,165,151 to Weiner teaches a relatively simple sedation delivery system that uses a microprocessor controller that commands a flow restriction clamp to adjust the diameter of an intravenous line based upon an input signal of a pulse oximetry device. The patient's blood oxygen saturation is measured by a pulse oximetry device and then compared with known limits. If the patient's blood oxygen saturation level exceeds a first limit or falls below a second lower limit the flow restriction clamp will alter sedative drug flow accordingly by manipulating the cross section area of the infusion conduit.

This device in its attempt to be simple has many shortcomings related to safety and efficacy. Particularly, this device lacks the ability to alert an attending physician of a change in sedation fluid flow rate and a change in the condition of the patient. Furthermore the above-mentioned device does not provide a means for a clinician to establish an initial drug delivery profile. This device fails to monitor many patient vital signs such as blood pressure, temperature, respiration rate, and capnography readings. A lone pulse oximetry device may not be able to accurately assess the true condition of the patient, leaving the patient vulnerable to improper controller adjustments of fluid flow rate.

A more complex infusion automation and delivery apparatus is described in U.S. Pat. No. 6,745,764 to Hickle. This device adjust the flow of sedative drugs to a patient by monitoring a plurality of patient physiological parameters and comparing these parameters to preset values. A controller actively evaluates the input from the patient monitors and changes fluid flow accordingly. The device although safe and effective, is designed for specific applications and practice settings; consequently much of the functionality of this device is unneeded in other simpler practice settings. Accordingly a need clearly exists for a device that provides safety to a patient that can be relatively inexpensive and portable.

### SUMMARY OF THE INVENTION

The present invention provides apparatuses and methods to safely and economically deliver infusion fluid to a patient during a medical procedure. The infusion fluid may be a sedative, analgesic, amnestic or other pharmaceutical agent (drug) for alleviating a patient's pain and anxiety before, during and/or after a medical or surgical procedure. In general the apparatus comprises a microprocessor-based controller that receives inputs from physiological monitors attached to a patient. The system controller processes the data from the physiological monitors and based upon a fluid infusion algorithm delivers infusion fluid to a patient. The physiological monitors monitor the patient throughout the course of the procedure and depending upon the health of the patient, drug delivery may be adjusted to optimize the procedure while ensuring the patient's health is maintained.

An additional aspect of this invention is directed toward monitoring the infusion delivery apparatus to ensure patient safety. Functionality detectors such as an occlusion sensor, air-in-line sensor and a fluid detection sensor alert a clinician to such hazards as a pressure build up in the infusion line, air-bubbles in the infusion line, and the absence of fluid in the infusion line. Upon detection of a hazard, system controller will adjust the flow of infusion fluid to mitigate the risk.

An additional aspect of this invention is directed toward alerting an attending clinician of a change in the patient's condition. If the health of a patient changes beyond a user-specified amount, the clinician may be alerted to the situation by way of audio and/or visual alert device. Furthermore, the clinician may be alerted to a system hazard as detected by the functionality detectors.

In accordance with the present invention, apparatus and methods are provided for improved automated delivery of sedative drugs to a patient. Safe and effective rates of infusion are provided by use of the system that monitors a plurality of patient parameters and adjusts the rate of infusion to an appropriate amount as decided by the clinician or system controller. Pre-established parameter ranges are provided before and/or during infusion of fluids into the patient. The infusion rate is continuously adjusted in response to increases or decreases in patient parameters compared to the pre-established parameter ranges. Attending clinicians are apprised of a patients condition at all times and are alerted to adverse patient responses to infused fluids and to actions taken by the system controller. Additional safety features include, line occlusion detection means, air-in-line detection means, and free flow prevention means.

### BRIEF DESCRIPTION OF THE DRAWINGS

FIG. 1 is an illustration of the apparatus of the present invention, including the controller and infusion delivery device.

FIG. 2 is a block diagram detailing a measuring system for determining the concentration of infused fluid, particularly propofol, in respired gases.

FIG. 3 is a block diagram illustrating a process for monitoring the pulse transit time of a patient being receiving infusion fluid.

FIG. 4 is a schematic illustration of the apparatus of the present invention, including a blood pressure cuff and pulse oximetry probe.

FIG. 5 is a schematic illustration of the apparatus of the present invention, including a blood pressure cuff and patient responsiveness device.

FIG. 6 is a schematic illustration of the apparatus of the present invention including two infusion delivery devices.

FIG. 7 is a block diagram depicting the flow rate logic utilized by the system controller.

FIG. 8 is a schematic illustration depicting the system controller used in conjunction with a status indicator, user interface and patient sensors.

FIG. 9-A is a schematic illustration of a first infusion delivery device in a first closed flow position.

FIG. 9-B is a schematic illustration of a first infusion delivery device in a second intermediate position.

FIG. 9-C is a schematic illustration of a first infusion delivery device in a third free flow position.

FIG. 10 is an illustration depicting the system controller used in conjunction with a wireless printer.

FIG. 11 is a block diagram depicting multiple system controllers used together under a central server.

FIG. 12 is a block diagram depicting the system controller used in conjunction with an external display.

### DETAILED DESCRIPTION OF PREFERRED EMBODIMENTS

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Further, unless otherwise indicated, the terms and expressions employed herein have been chosen for the purpose of describing the illustrative embodiments of the present invention for the convenience of the reader and are not for the purpose of limiting the invention.

Further, it is understood that any one or more of the following-described embodiments, expressions of embodiments, examples, etc. can be combined with any one or more of the other following-described embodiments, expressions of embodiments, examples, etc.

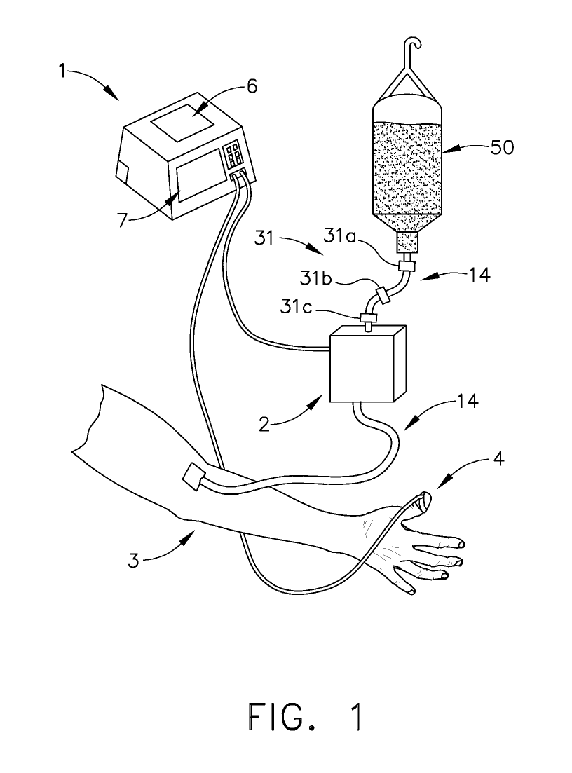

The system illustration of FIG. 1 depicts a first expression of the system architecture of the invention. The diagram illustrates the relationship between system controller 1, infusion delivery means 2 and a fluid source 50, as well as other subsystems of note, each of which is described in more detail later. Infusion delivery means 2 delivers infusion fluid from fluid source 50 to patient 3 based upon control signals issued by system controller 1. Infusion fluids administered to patient 3 may include but are not limited to chemical agents, analgesics, anesthetics, blood, plasma, antibiotics, crystalloids, saline, and colloids. System controller 1 receives information related to the patient's health by way patient sensors 4. Patient sensors 4 may comprise one or a plurality of patient sensors including, but not limited to devices that monitor pulse oximetry, blood pressure, capnography, electrocardiogram (ECG), electroencephalogram (EEG), respiration rate, temperature, patient responsiveness, concentration of respired gases in the blood stream, and perceptive pain assement.

Pulse oximetry sensors (such as The Voyager manufactured by Dolphin Medical) are provided for trans-illumination of a blood-perfused portion of the body to measure light extinction during trans-illumination as is known in the art. The sensor is typically mounted on either a fingertip or earlobe and conforms to the contours of the patient's body.

Blood pressure monitors and blood pressure cuffs (Advantage Mini from SunTech Medical Instruments) are comprised of an inflatable fabric cuff that when inflated constricts blood flow through a patient's arm. The cuff measures the periphery blood vessels pressure and the cuff then provides a patient's systolic, diastolic, and mean arterial blood pressure. An alternative embodiment involves a blood pressure cuff mounted on the patient's wrist. A LifeWise™ Wrist-Cuff Blood Pressure Monitor could easily be adapted to monitor a patient's blood pressure readings and send a corresponding signal to system controller 1.

Capnography modules such as the CO₂ WaveForm Analyzer from Cardiopulmonary Technologies used in conjunction with a standard oral-nasal cannula as are well known in the art, allow for collection of respired gases from a patient and an analysis of respiratory carbon dioxide concentration. An oral-nasal cannula is preferably positioned adjacent to the nose and mouth of a patient to receive the patient's respired breath. Excessive percentage of CO₂ found in a patient's respired breath might indicate an adverse reaction to infused fluids as is well understood by those skilled in the medical arts. The capnography module in combination with the oral-nasal cannula may also be used to monitor a patient's respiration rate by measuring the time between peak values as recorded by a pressure transducer or other means.

Electrocardiogram (ECG) modules may consist of an M12A Front-End (FE) module; differential converter circuit and Receiver chip (11005-012-50 Rev A1 from Mortara). Electrodes attached to the patient emit and receive electrical pulses to diagnose heart rate and vascular disorders. ECG modules are used to measure the rate and regularity of heartbeats, the size and position of the chambers, the presence of any damage to the heart, and the effects of drugs.

Electroencephalogram (EEG) modules are comprised of a plurality of electrodes fixed to a patient's head to detect electrical activity of the brain. This electrical activity can indicate information such as brain activity levels and neural disorders. Furthermore, an EEG device may be used to measure a patient's respiratory rate by a technique known as transthoracic impedance (TTI). In TTI, EEG electrodes are attached to a patient's trunk. Electrical signals are sent from the electrodes and the time required for the signals to return to the electrodes is measured. The difference in time can be indicative of the oxygen content of the patient's body, particularly the lungs. Consequently, respiration rate may be determined by taking a plurality of EEG measurements over a period of time.

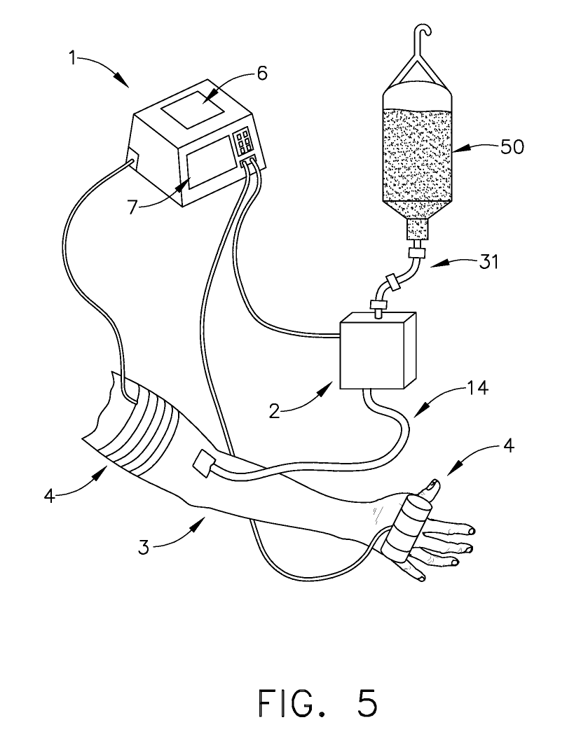

A patient responsiveness device, similar to the device disclosed in U.S. patent application Ser. No. 10/791,959 to Katz and Nesbitt, may be used in conjunction with the above-mentioned sensors. This device comprises a query initiate device and a query response device. The patient response system operates by obtaining a patient's attention with the query initiate device and commanding the patient to activate the query response device. The query initiate device may be any type of stimulus such as a speaker via an earpiece, which provides an auditory command to a patient to activate the query response device. The query response device may be a hand piece that can take the form of for example, a toggle or rocker switch or a depressible button or other movable member hand held or otherwise accessible to the patient so that the member can be moved or depressed by the patient upon the patient's receiving the auditory or other instruction to respond. Alternatively, a vibrating hand mechanism may be incorporated into the hand piece that cues the patient to activate the query response device. In one embodiment, the query initiate device is a cylindrical handheld device containing a small 12VDC bi-directional motor enabling the handheld device to vibrate the patient's hand to solicit a response (FIG. 5)

System controller 1 may serve to monitor the time delay between a signal generated by the query initiate device and a patient's response as recorded by query response device. An excessive time delay from the query to the response may indicate that a patient is experiencing an adverse reaction to the infused fluid, particularly if the infused fluid is a sedative and the patient is becoming over sedated. The time may be compared to a predetermined threshold value and if found to be outside an appropriate time range, system controller 1 will command infusion delivery means 2 to adjust the fluid flow rate through IV line 14 to a more acceptable range.

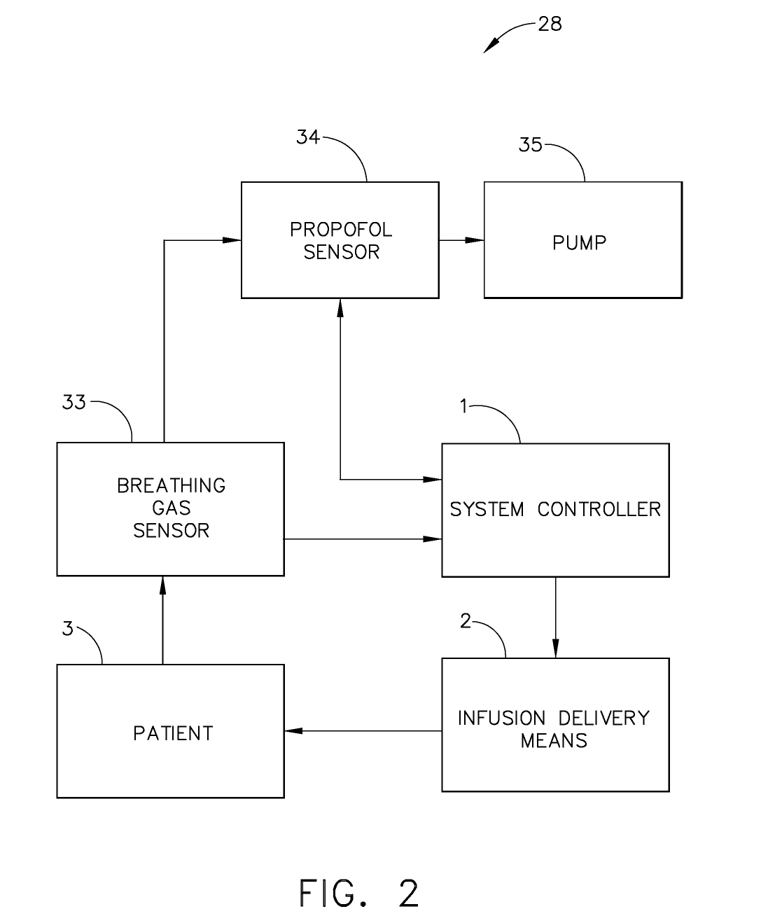

In a second expression of the invention, a breath analyzer 33 is incorporated as part of the invention to detect the concentration of an infused fluid, for example, propofol, in a patient's blood stream as described in US20050022811 to Kiesele et al. As shown in FIG. 2, breathing gas sensor 33 is fluidly connected to a patient's airway and electrically connected to system controller 1. Breathing gas sensor 33 may be a CO2, O2 volume flow or temperature sensor to measure characteristics of a patient's respiratory gases. Propofol sensor 34 located downstream of breathing gas sensor 33 also receives exhaled patient gases. Propofol sensor 34 is further fluidly connected to a downstream pump 35. Propofol sensor 34 may be an electrochemical gas sensor, SAW (Surface Acoustic Wave) sensor, ion mobility sensor, a gas chromatography, mass spectrometer, or a combination of a gas chromatograph and an ion mobility or mass spectrometer. System controller 1 is connected with propofol sensor 34 and pump 35, so that system controller 1 actuates pump 35 for a sampling breathing gas depending on the signal of breathing gas sensor 33. Propofol sensor 34 sends a measured signal for concentration of propofol to system controller 1.

In an alternate embodiment of the second expression, breathing gas sensor 33 receives respiration parameters from system controller 1 and actuates pump 35 such that propofol sensor 34 measures (for example) the end tidal propofol concentration in the respiratory flow breathed out. The mode of operation in the measuring system is such that depending on the measured signal of breathing gas sensor 33, which is especially a CO2 sensor, pump 35 is actuated by system controller 1, so that samples reproducible in respect to the propofol content, especially of alveolar air, are delivered for the propofol measurement from the respiratory flow.

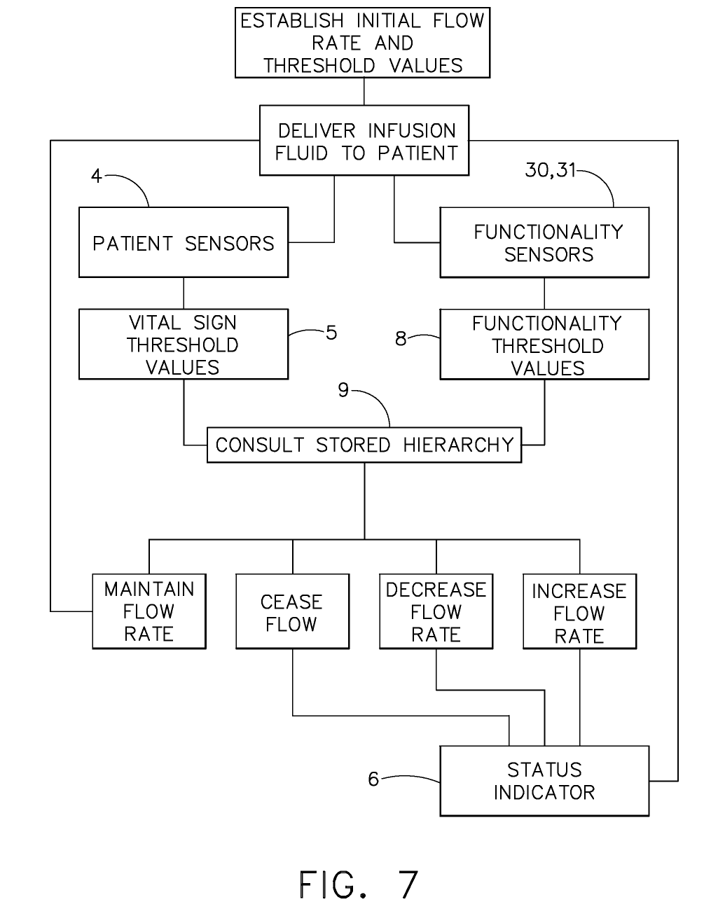

In still another alternate embodiment of the second expression, system controller 1 monitors a patient's respired gases in the event a patient sensor 4, such as for example, a responsiveness monitor, indicates a patient is sedated or when a patient's blood oxygen saturation falls below a vital sign threshold value 5 (FIG. 7). The concentration of propofol as measured by propofol sensor 34 will be used as a baseline value. Subsequent measurements that indicate the propofol concentration is greater than the baseline value will prompt system controller 1 to reduce the flow of infused fluids (in this example, propofol) to the patient 3.

A third expression of the invention includes means for assessing arousal, pain and stress during fluid infusion. As described in US2004/0015091 to Greenwald and Dahan, ECG electrodes and a photo-plethysmography (PPG) device may be used concurrently to generate a Pulse Transit Time (PTT) value that may be interpreted to evaluate the patient's consciousness as well as stress and pain levels.

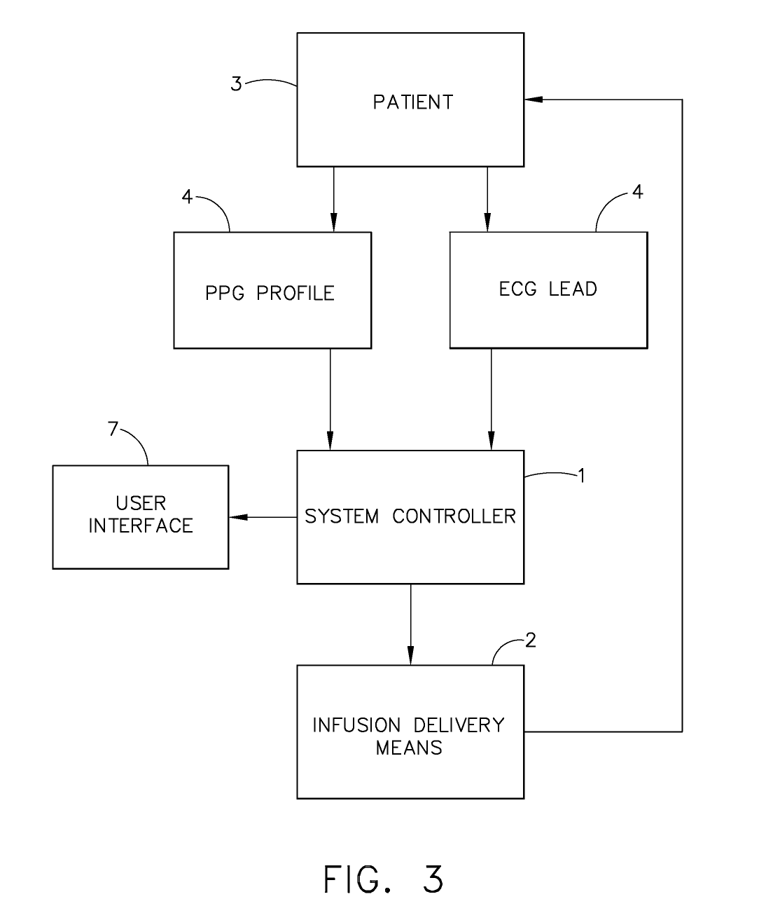

As shown in FIG. 3, system controller 1 continuously monitors ECG and PPG waveforms, both monitors are represented by item 4. For each cardiac cycle, fiducial points are identified to indicate the pulse onset time (as measured by ECG) and pulse arrival time (as measured by PPG). The onset and arrival times for each cardiac cycle are paired, and the time difference or pulse transit time (PTT) is the interval estimate for that beat. System controller 1 monitors trends in PTT values for a rapid decrease or increase. A rapid decrease will result in system controller 1 prompting infusion delivery means 2 to provide supplemental infusion fluid to patient 3, while a rapid increase in PTT will result in system controller 1 prompting infusion delivery means 2 to reduce the flow of infusion fluid to patient 3.

An alternate embodiment of the third expression incorporates an entropy module, such as for example, the S/5 Entropy Module developed by Datex-Ohmeda Division, Instrumentation Corp. As described in US20030055355, the entropy module monitors the change in entropy of an EEG signal. Interpretation of an entropy level can give a clinician an indication of the depth of anesthesia of a patient. A high level of signal entropy indicates a patient is fully awake and alert, conversely, as the entropy level approaches zero, a patient is entering a deep level of anesthesia. An entropy module may be incorporated into system controller 1 or may be electronically connected to system controller 1. In either case, system controller 1 will evaluate the trend in entropy level and will prompt infusion delivery means 2 to alter the flow of infusion fluid to patient 3 accordingly.

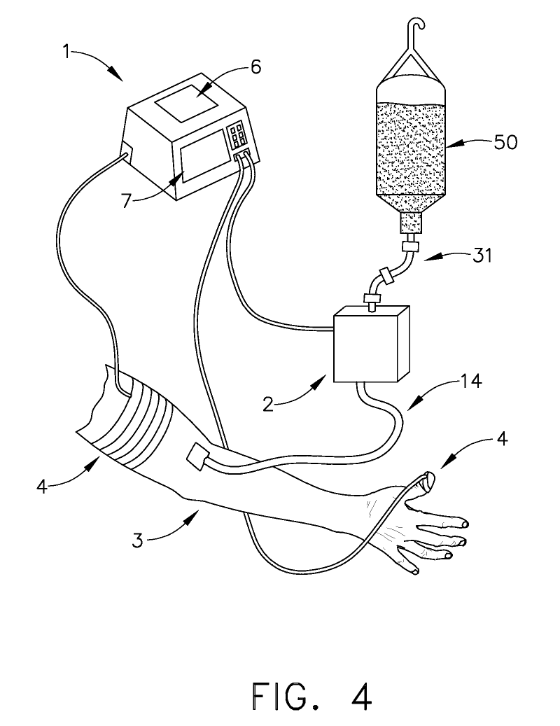

A combination of the above devices may be used to provide a more sound evaluation of the patient's condition. In one expression, patient sensors 4 comprise a pulse oximetry sensor that measures the percentage of oxygen found in a patient's bloodstream and a non-invasive blood pressure sensor for measuring a patient's systolic and diastolic blood pressure. It is understood in the art that measuring a patient's blood oxygen saturation and blood pressure provides an indication of the relative health of a patient. Blood pressure and blood oxygen saturation levels are of particular importance in assessing the effects of sedative drugs upon a patient. Various patient sensor 4 combinations are shown to illustrate the modularity of the current device. As an example, FIG. 1 depicts the current invention with a lone pulse oximeter sensor, while FIG. 4 depicts the current invention with a both a pulse oximeter sensor 4 and blood pressure device 4. Other combinations of sensors can be used such as a blood pressure cuff 4 and a patient responsiveness monitor 4 as shown in FIG. 5.

Now referring to FIG. 1, a fourth expression of the invention includes detectors in IV line 14 for detecting the presence or absence of infusion fluid in IV line 14. A fluid detection sensor 31a continuously monitors IV line 14 for the presence of infusion fluid while infusion delivery means 2 is active. Upon sensing an absence of fluid in IV line 14, a signal is sent to system controller 1 which in turn halts further delivery of infusion fluid and alerts the attending clinician by way of status indicator 6. The fluid detection sensor 31a may be any of a number of different types of sensors including but not limited to optical sensors, ultrasonic sensors, proximity sensors, or electromagnetic sensors.

An air-in-line sensor 31b monitors IV line 14 for the presence of air bubbles, which may present a danger if air bubbles reach the patient's bloodstream. The air-in-line sensor 31b may be any number of different sensor types including optical and ultrasonic sensors. The sensor periodically sends a signal to system controller 1 describing the air content of IV line 14. This command indicates the amount of air detected in the line over a particular time period. Alternatively, the air-in-line sensor 31b may register an air bubble greater that a predetermined maximum volume. Upon receiving a signal from the air-in-line sensor 31b, system controller 1 will compare the signal with a predetermined threshold value. System controller 1 may maintain, increase, decrease, or halt the flow of fluid similar in a manner similar to that described above relating to sensors 4.

The current invention may also include means to detect an occlusion or blockage in IV line 14. Occlusions pose a risk to the patient in that if the blockage is removed, a sudden bolus of infusion fluid may reach the patient. If the blockage is not removed and pressure continues to increase, IV line 14 or a blood vessel may rupture. To circumvent this situation, an occlusion sensor 31c, which may be a strain gauge, piezoelectric, or other type of pressure transducer continuously monitors the pressure of IV line 14. The occlusion sensor 31c sends an output signal to system controller 1 regarding the pressure of IV tube 14. System controller 1 will compare the value of the occlusion sensor 31c with a predetermined pressure threshold. System controller 1 will in turn send an appropriate command to infusion delivery means 2 to reduce or cease the fluid flow.

The occlusion senor 31c, air-in-line sensor 31b, and fluid detection sensor 31a all serve to monitor the functionality of infusion delivery means 2. These three sensors will collectively be referred to herein as functionality detectors 31, and are schematically depicted in FIG. 1.

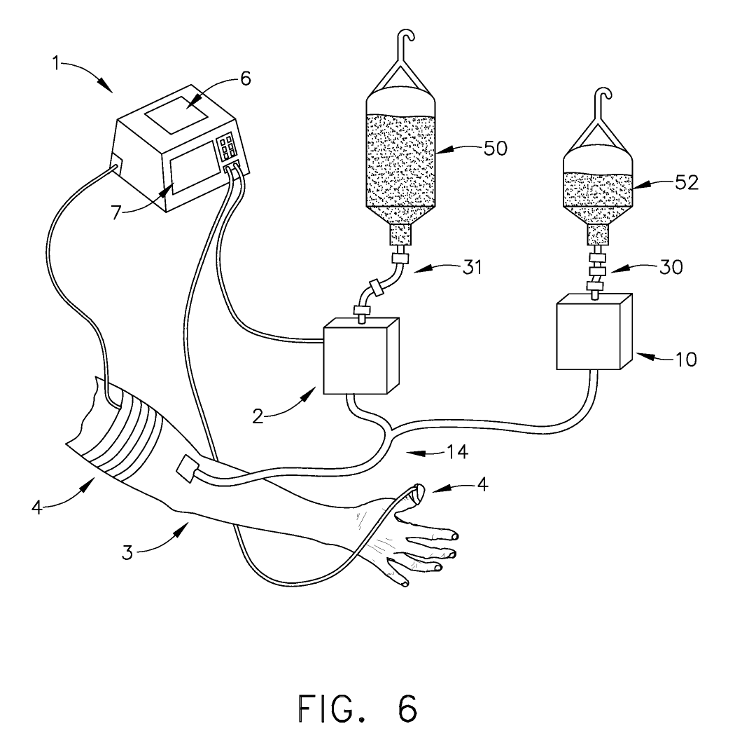

Now referring to FIG. 6, a fifth expression of the invention includes the capability to deliver two or more infusion fluids 50 and 52 to a patient simultaneously. In a first embodiment, the alternative infusion fluid(s) will be supplied to patient 3 by way of alternate infusion delivery means 10. Infusion delivery means 2 delivers a first infusion fluid from fluid source 50 to patient 3 while alternate infusion delivery means 10 supplies a second infusion fluid from fluid source 52. Alternate infusion delivery means 10 like infusion delivery means 2 may be a gravity feed device or a fluid pump as described later. All functionality associated with infusion delivery means 2 may be duplicated with such devices as an alternate occlusion detector, alternate free-flow detector, and alternate air-in-line detectors, referred to collectively as alternate functionality detectors 30. All outputs of alternate functionality detectors 30 are transmitted to system controller 1 which evaluates sensors 4, functionality detectors 31, and alternate functionality detectors 30 to regulate the rate of fluid infusion.

As shown in FIG. 3, a clinician may establish an initial infusion profile by programming system controller 1 by way of user interface 7 (FIG. 1). An infusion profile may include the type of fluid to be infused, initial bolus of fluid, maintenance rate, total amount of fluid to be infused, average rate of infusion, and total infusion time. In a second embodiment, a clinician may choose an infusion profile from a stored group of infusion profiles. In addition to setting an infusion profile, a clinician may enter information about the patient by way of user interface 7 and a suggested infusion profile will be calculated based upon patient information and a pre-programmed pharmacological model. After calculation of the suggested infusion profile, the clinician will have the opportunity to reject or allow the infusion profile by indicating so on user interface 7. The technique of infusing fluids into a patient to achieve a desired effect-site concentration is known as target controlled infusion (TCI) and is well understood in the sedation and anesthesia arts. An alternative infusion delivery algorithm that may be employed in the current invention is found in U.S. application Ser. No. 10/886,255 filed Jul. 7, 2004, which discloses a drug delivery algorithm for use in an automated infusion delivery device.

An alternative to a pre-programmed infusion profile is a patient controlled fluid delivery device. Patient controlled analgesia, and patient controlled sedation are well known in the infusion delivery arts and are easily incorporated into the current invention.

After entering an infusion profile, a clinician may enter patient threshold values into system controller 1. Patient threshold values 5 (FIG. 7) are numeric values representing patient vital signs and are electronically stored in system controller 1. A lower and upper patient threshold value 5 may be set for each physiological parameter measured by patient sensors 4. For instance, an upper threshold value of 135/90 mm HG may be set for a blood pressure sensor while the lower threshold value may be 90/50 mm HG. Furthermore, functionality threshold values 8 may be entered into system controller 1. Functionality threshold values 8, similar to patient threshold values 5, provide an upper and lower limit for functionality detectors 31 and alternate functionality detectors 30. In an alternate embodiment, system controller 1 may automatically generate threshold values 5,8. These values are based upon pre-programmed algorithms contained in system controller 1. A clinician may prompt system controller 1 to generate threshold values 5 and 8, then the clinician may approve, reject, or modify the generated threshold values 5,8.

Upon establishing the initial flow profile and threshold values 5 and 8, system controller 1 will prompt infusion delivery means 2 and alternate infusion delivery means 10 to begin delivering infusion fluid to patient 3. As infusion fluid is being delivered, patient sensors 4, functionality sensors 31, and alternate functionality sensors 30 monitor their respective fields. Data from sensors 4, 30, and 31 are transmitted to system controller 1 for further analysis. Data received from sensors 4 are compared against vital sign threshold values 5. Similarly, data received from functionality sensors 30 and 31, are compared against functionality threshold values 8.

System controller 1 will issue commands to infusion delivery means 2 and 10 to maintain or alter the infusion fluid delivery profile based upon comparisons between sensors 4,30 and 31 with threshold values 5 and 8. These command are an attempt to affect the vital signs of patient 3 and the operating parameters of the infusion delivery means 2 and 10. Infusion delivery means 2 and 10 are devices that physically induce or prohibit the flow of infusion fluid through IV line 14 into patient 3. The commands issued by system controller 1 to infusion delivery means 2 and 10 may be to increase, maintain, decrease, or cease the current flow of infusion fluid into patient 3.

System controller 1 is a typical electronic controller that is well understood in the art. System controller 1 has the capability to receive multiple input signals from an external source such as sensors 4 and to analyze these signals with a microprocessor. Output signals are issued based upon a predetermined software response to particular input signals. The software included in system controller 1 has predefined threshold limits of patient parameters. An input signal above an upper threshold limit will induce system controller 1 to produce an output signal commanding infusion delivery means 2 and 10 to increase the flow of infusion fluid to patient 3. Likewise, an input signal below a lower threshold limit will induce system controller 1 to produce an output signal commanding infusion delivery means 2 and 10 to decrease or cease the flow of infusion to patient. An input signal that is neither below the lower threshold limit nor above the upper threshold limit will induce system controller 1 to maintain the current flow of infusion fluid into patient 3. Examples of medical controllers that are sold today, which could easily be adapted for use in the current invention include; the Cancion CRS Therapy from ORQIS Medical, The Avant® 2120 sold by Nonin Medical, and the Vital Signs Monitor 300 Series from Welch Allyn.

System controller 1 allows a clinician to establish a threshold hierarchy 9 whereby the actions of system controller 1 in response to sensors 4, 30, and 31 are governed in a particular manner. For example, if more than one sensor is in use, a clinician may program system controller 1 to alter infusion delivery means 2 only if all the sensors 4 report patient parameters outside a threshold value. Alternatively, particular patient or system parameters may be given a higher priority than others, where only a subset of sensors 4, 30, and 31 report a patient or system parameter outside of a threshold value is sufficient alter infusion delivery means 2. Furthermore, system controller 1 may be programmed in a multitude of other ways depending upon clinician preference, which will be obvious to those skilled in the art.

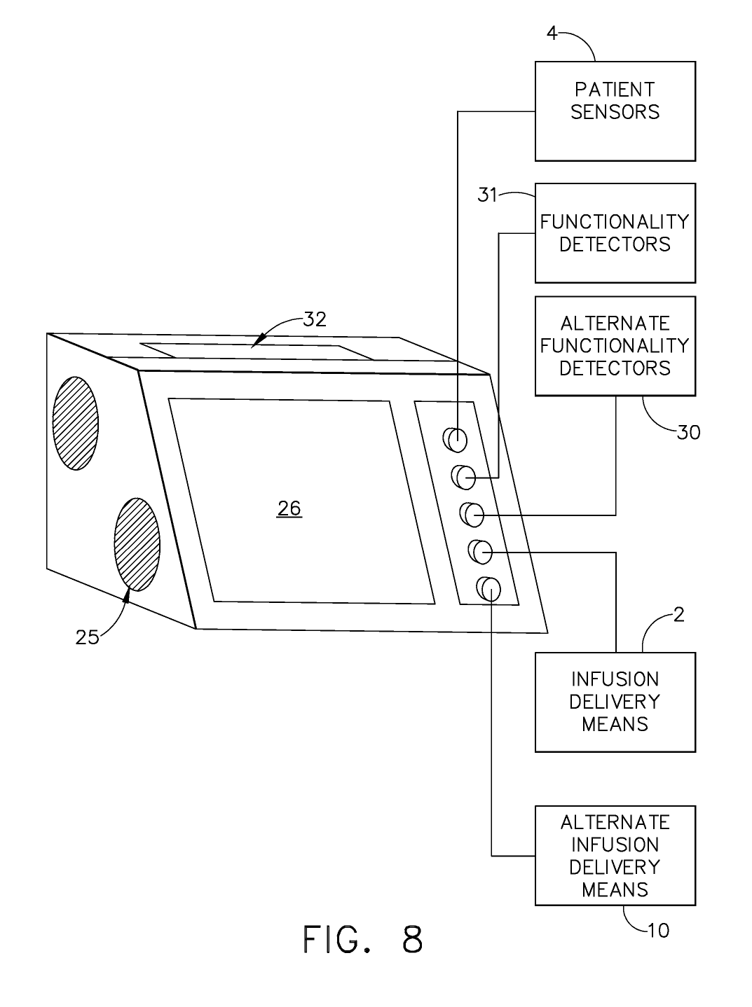

Now referring to FIG. 8, system controller 1 may further include a user interface 7 (FIG. 1) to allow a clinician to adjust settings and parameters associated with system controller 1. User interface 7 (FIG. 1) also includes means to display operating parameters to the clinician indicating the status of system controller 1, patient 3 and infusion delivery means 2. In a preferred embodiment, user interface 7 is an LCD touchscreen 26. LCD touchscreen 26 has both the ability to display patient and system operating parameters and at the same time allow a clinician to provide input into system controller 1.

Status indicator 6 is a module electrically connected to system controller 1 that alerts an attending physician of a change in a multitude of operating parameters measured by the current invention. In the event the patient's physiological parameters reach a dangerous level, status indicator 6 will alert an attending clinician to the patient's condition and any corrective action already taken by system controller 1. In certain circumstances, status indicator 6 will alert a clinician to a patient condition requiring clinician intervention.

In a first embodiment, status indicator 6 is a light bar 32 comprised of a plurality of LED lights as shown in FIG. 8. Light bar 32 may produce a first color to indicate a change in patient condition; a second color to indicate an action by system controller 1, and a third color to indicate that clinician intervention is required. Additional colors may be used to indicate further changes and operating conditions. A second embodiment comprises an audio output device 25, such as a speaker or earphone, which produces a unique sound for situations such as a change in patient condition, an action taken by system controller 1, a request for clinician intervention and other system actions. The unique sound may be a pre-recorded voice apprising the clinician of the patient's status, and suggesting a course of action. In a third embodiment, text messages are displayed to the user by way of LCD touchscreen 26, providing detailed information regarding patient's 3 condition and the current actions of system controller 1 by way of LCD touchscreen 26. It should be noted that two or more of the embodiments mentioned above might be combined to provide multiple indicia of patient and system conditions. As an example, status indicator may flash a light, emit a sound and display a text message to alert a clinician to a change in patient status. Furthermore, the severity of a change may dictate what means status indicator 7 uses to alert a clinician. A loud audio alert, and several flashing lights may signify life-threatening events, while a soft chirp from audio output device 25 may represent a minor change in patient condition.

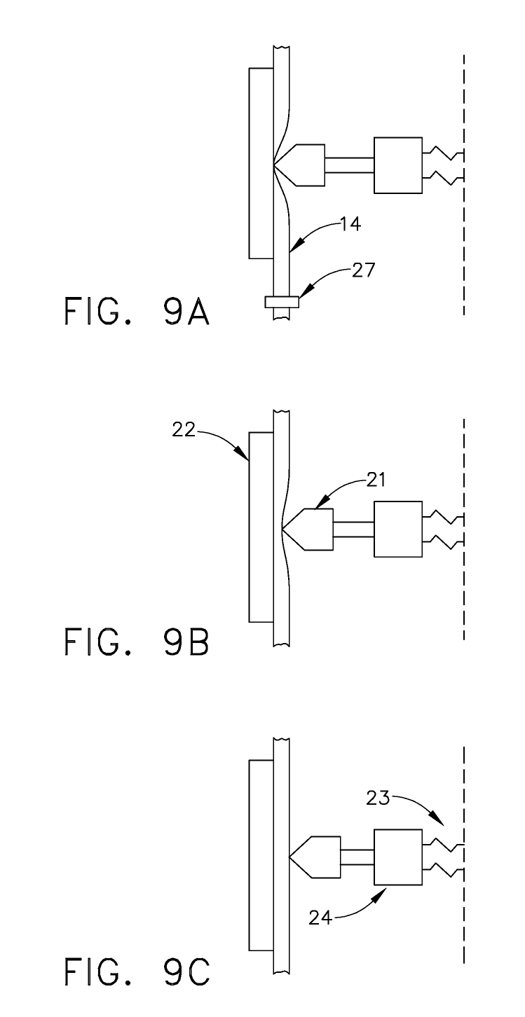

In a first embodiment, infusion delivery means 2 is a gravity feed mechanism which utilizes a variable pressure clamp 20 to contact IV line 14 and physically reduce the cross sectional area of IV line 14 as shown in FIGS. 9 A-C. Variable pressure clamp 20 consists of two opposably mounted rigid bodies whereby variable body 21 is capable of lateral motion with respect to fixed body 22 as shown in FIG. 9. Movement of variable body 21 is made possible by bi-directional motor 24. Bi-directional motor 24 receives operating commands from system controller 1 in the form of voltage signals. FIG. 9-A depicts variable pressure clamp 20 in a first closed flow position where IV line 14 has an original cross sectional area of approximately zero. FIG. 9-B depicts variable pressure clamp 20 in a second intermediate position. Bi-directional motor 24 has increased the distance between variable body 21 and fixed body 22 whereby the cross sectional area of IV line 14 is increased by a predictable amount. Finally FIG. 9-C depicts variable pressure clamp 20 in a third free flow position. In this position, bi-directional motor 24 has moved variable body 21 even farther away from fixed body 22, whereby IV line 14 is completely unobstructed allowing unimpeded flow through IV line 14.

Although only three fluid flow positions are shown, bi-directional motor is capable of finely tuning the distance between variable body 21 and fixed body 22 to produce many different fluid flow rates. Furthermore, bi-directional motor may move variable body 21 closer to fixed body 22 to decrease the flow rate of infusion fluid as directed by the clinician and system controller 1. In an alternate embodiment, bi-directional motor 24 may be replaced by a manual engagement knob, which would allow a clinician to manually adjust the amount of engagement between variable body 21 and IV line 14.

In a first embodiment, variable pressure clamp 20 contains biasing springs 23 which have a spring constant sufficiently high to ensure variable body 21 is biased toward a default closed flow position as shown in FIG. 9-A. Biasing springs 23 are included to serve two purposes, the first being a means to prevent inadvertent fluid flow through IV line 14, the second being a means to compensate for the variances in manufacturing tolerances. Infusion fluid allowed to flow freely to the patient 3, may present a health hazard. Biasing variable pressure clamp 20 to the default fluid flow rate to zero mitigates the possibility of experiencing a free flow condition. Defaulting the initial fluid flow to zero will calibrate the fluid flow rate as the bi-directional motor 24 retracts variable body 21 by a known distance.

Similarly, biasing springs 23 will compensate for small variances in tolerances that may lead to a fluid flow rate out of calibration. In a first embodiment system controller 1 issues commands to bi-directional motor 24 to adjust the position of variable body 21 but does not rely on a sensor to detect the rate of fluid flow through IV line 14. Without biasing springs 23, there is no insurance that variable body 21 is positioned close enough to fixed body 22 to ensure that fluid flow through IV line 14 is zero in the default position. This may lead to fluid being supplied to the patient inadvertently and unexpectedly, which may put the patient at risk. Furthermore, the fluid flow rate through IV line 14 will not be known as the variable body 21 retracts from fixed body 22.

In a second embodiment, fluid flow meter 27 (FIG. 9-A) may be introduced into the IV line 14 downstream of variable pressure clamp 20 to monitor the volumetric flow rate, mass flow rate, flow velocity or other flow characteristics through the line and allow system controller 1 to adjust the distance between variable body 21 and fixed body 22 accordingly. Either an inline flow meter or an insertion flow meter may be used as is well known in the art. The output of fluid flow meter 27 is sent to system controller 1 which will adjust infusion delivery means 2 to ensure the preferred fluid flow rate is being supplied to the patient 3.

In a third embodiment, infusion delivery means 2 is a peristaltic type pump. A peristaltic pump utilizes a row of peristaltic fingers that sequentially compress and uncompress IV line 14 to create a wavelike motion to induce fluid flow through IV line 14. The speed of peristaltic motion is governed by voltage signals delivered to infusion delivery means 2 by system controller 1. In the current invention line 14 is removably attached to fluid reservoir at one end and removably attached to patient 3 at the opposite end. Ideally, IV tubing 14 is a segment of tubing specifically adapted for use with a peristaltic pump that may endure a series of deforming impacts and still maintain the original fluid flow properties and flexibility of a line that has not been subject to deforming impacts. Alternatively many alternative pumps may be used in place of a peristaltic pump, including but not limited to, bellows, diaphragm, piston, syringe, roller, lobe, and oscillating pumps.

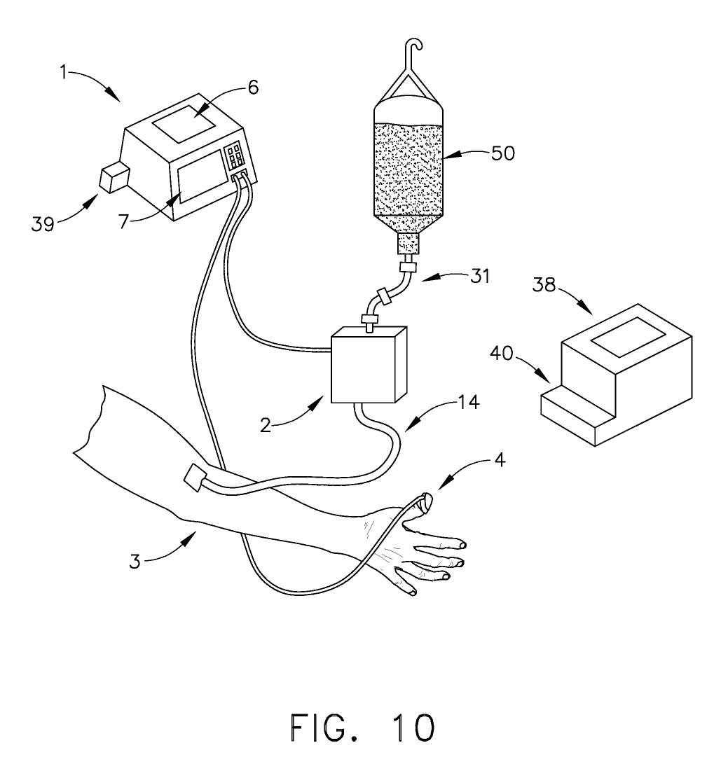

Now referring to FIG. 10, the current invention may be adapted to interface with wireless printer 38. In a first embodiment, system controller 1 transmits relevant data to wireless printer 38 by way of an integrated wireless transmitter 39. Wireless transmitter 39 may incorporate either an IEEE 802.11 or Bluetooth type technology. Similarly, wireless printer 38 receives data transmitted from system controller 1 with a wireless receiver 40. Wireless receiver 40 may be either electrically connected or fully integrated with wireless printer 38. In the event where multiple wireless printers are found in a single location, a clinician may select which printer system controller 1 communicates with. Options include, printing to the printer with the strongest wireless signal strength or printing to a designated printer.

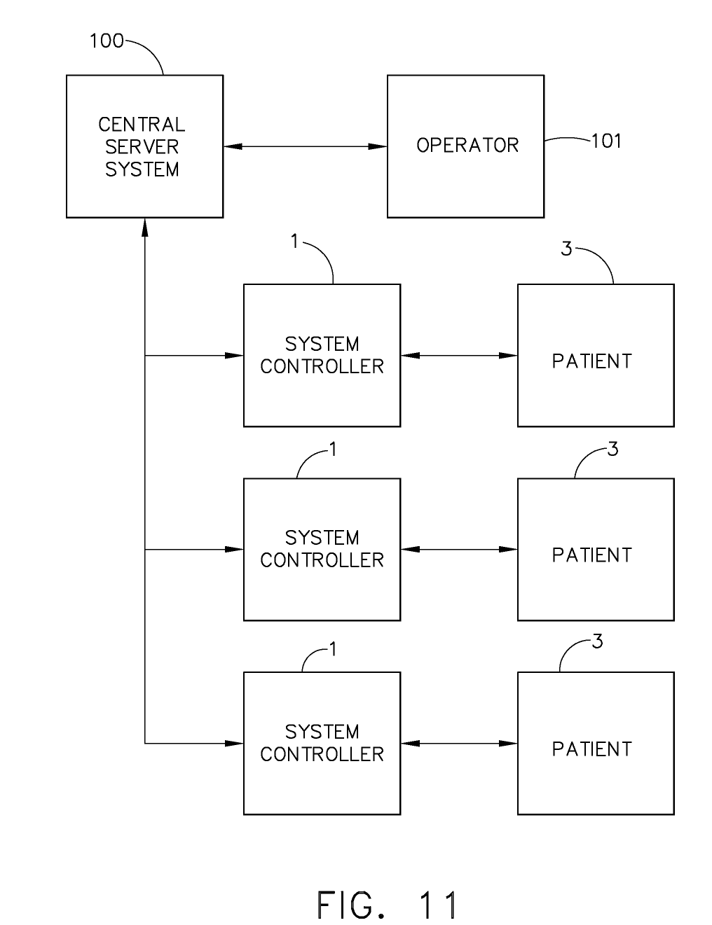

Another implementation of the current invention includes system controller 1 wirelessly communicating with central server system 100 as seen in FIG. 11. Central server system 100 is a typical computer server such as an IBM Cluster 1350 xSeries 346 or a HP Integrity RX8620-32 server, which receives information regarding a patient's condition and operating parameters of system controller 1. Central server system 100 resides is a second room location and is capable of receiving and processing data from multiple system controllers located throughout a health care facility. Server user interface 101 allows a clinician or operator to monitor the various system controllers reporting to central server system 100 and to operate the system controllers remotely. This allows a single clinician to monitor multiple system controllers reducing the number of skilled personnel needed to effectively monitor a patient care center.

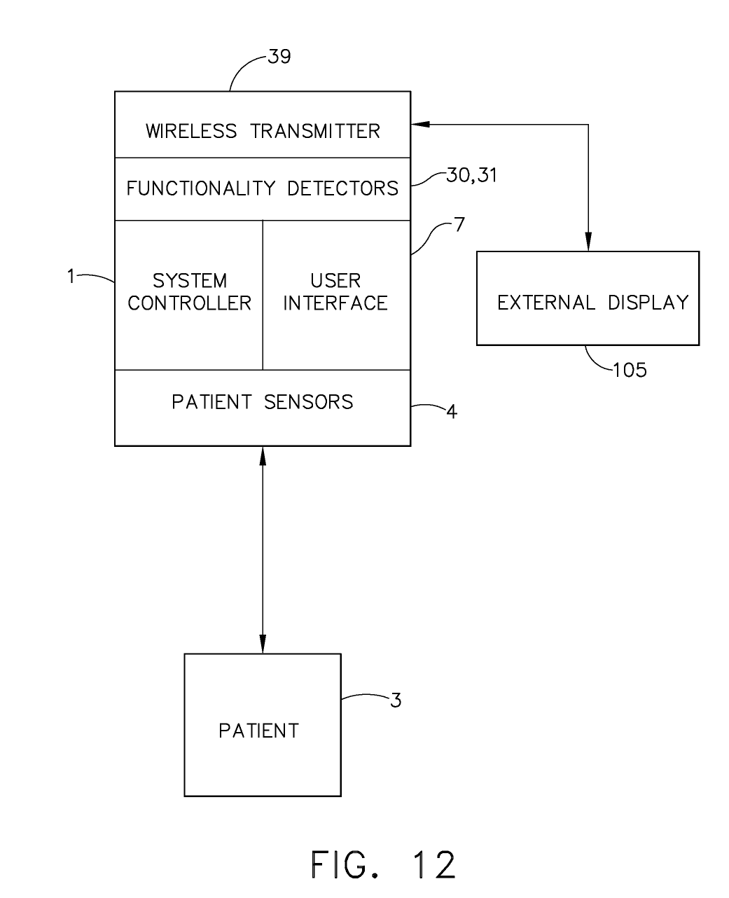

Now referring to FIG. 12, an external display 105 may be used in conjunction with system controller 1. External display 105 may be a LCD or cathode ray display device, such as for example, the MFGD 5621HD Display form Barco. In a first embodiment, external display 105 communicates with system controller 1 by way of wireless or infrared technology. System controller 1 utilizes wireless transmitter 39 to transmit data to external display 105. Data to be transmitted may include, information pertaining to the health of patient 3, information pertaining to the operation of the device as described by functionality detectors 30 and 31. Furthermore, the output of status indicator 6 may be duplicated by external display 105.

The interface between external display 105 and system controller 1 allows for a periodic verification of connection. In a first embodiment, external display sends a signal to system controller 1 indicating either an error is present in external display 105 or that no error is present in external display 105. An error may include conditions where external display 105 is not functioning properly. Upon receiving an error signal, system controller 1 will take appropriate action, which may include reducing the flow of infusion fluid to patient 3 and/or alerting a clinician of the change in status. A clinician may specify what action is to be taken by entering threshold values 5 (FIG. 7) into system controller 1 by way of user interface 7. Additional signals to be sent from external display 105 including an identifier unique to external display 105 whereby system controller 1 will recognize the identifier and associate all data from a particular identifier with a particular external monitor 105.

While aspects, embodiments and examples, etc. thereof, it is not the intention of the applicants to restrict or limit the spirit and scope of the appended claims to such detail. Numerous other variations, changes, and substitutions will occur to those skilled in the art without departing from the scope of the invention. For instance, system controller and components thereof of the invention have application in robotic assisted surgery taking into account the obvious modifications of such systems and components to be compatible with such a robotic system. It will be understood that the foregoing description is provided by way of example, and that other modifications may occur to those skilled in the art without departing from the scope and spirit of the appended claims.

We claim:

1. A system for providing an infusion fluid to a patient undergoing a medical or surgical procedure, the system comprising:
a. a plurality of physiological monitors adaptively coupled to the patient and generate corresponding signals representing the condition of the patient;
b. a gravity fed supply source of the infusion fluid;
c. a flow regulator for controlling a supply of the infusion fluid;
d. at least one functionality detector adapted to generate a corresponding signal representing the condition of the supply of the infusion fluid; and
e. an electronic controller interconnected between the plurality of physiological monitors and the at least one functionality detector and the flow regulator; wherein the electronic controller regulates the flow regulator in response to the signals from the plurality of physiological monitors and the at least one functionality detector.

2. The system of claim 1, wherein the plurality of physiological monitors is chosen from the group consisting of pulse oximetry, blood pressure, capnography, electrocardiogram, electroencephalogram, respiration rate, temperature, patient responsiveness, concentration of respired gases and pain.

3. The system of claim 1, wherein the functionality detector is chosen from the group consisting of a fluid detection sensor, an air-in-line sensor and a occlusion sensor.

4. The system of claim 1 further comprising a second supply source of an infusion fluid.

5. The system of claim 1, wherein the controller comprises means for inputting an infusion profile into the controller.

6. The system of claim 5, wherein the input is selected from the group consisting of type of fluid for infusion, initial bolus of fluid, maintenance rate, total amount of fluid for infusion, average rate of infusion and total infusion time.

7. The system of claim 1, wherein the controller comprises means for inputting patient threshold values into the controller.

8. The system of claim 1 further comprising an alarm indication selected from the group consisting of lights, text messaging and audio.

9. The system of claim 1, wherein the flow regulator is a variable pressure clamp.

10. The system of claim 9, wherein the variable pressure clamp is spring biased closed.

11. The system of claim 1, wherein the flow regulator is a peristaltic pump.

12. The system of claim 1 further comprising a wireless printer.
